(19) United States
(12) Patent Application Publication
(10) Pub. No.: US 2017/0143920 A1
(43) Pub. Date: May 25, 2017

(54) TRACHEAL TUBE

(71) Applicant: TERUMO KABUSHIKI KAISHA., TOKYO (JP)

(72) Inventors: Anri FUJII, Kanagawa (JP); Yasuyuki HONMA, Kanagawa (JP)

(21) Appl. No.: 15/331,521

(22) Filed: Oct. 21, 2016

(30) Foreign Application Priority Data
Nov. 19, 2015 (JP) 2015-226603

(51) Int. Cl. A61M 16/04; A61M 25/10

(52) U.S. Cl. CPC A61M 16/0445; A61M 16/0465; A61M 16/0443; A61M 25/1034; A61M 2207/00

(57) ABSTRACT

There is provided a tracheal tube that allows improvement in safety. The tracheal tube includes a tube main body, and a cuff that is mounted to the tube main body and has a barrel portion capable of being deflated and inflated. A covering region that covers a tracheal inner wall of a trachea in a state in which the barrel portion is inflated in the trachea has a sealing part that gets contact with the tracheal inner wall seamlessly in a circumferential direction of the barrel portion. The width of at least part of the sealing part is smaller than 1 mm.

### CROSS REFERENCE TO RELATED APPLICATIONS

This application is based on and claims priority to Japanese Application No. JP2015-226603, filed on Nov. 19, 2015, the entire contents of which are incorporated herein by reference.

### BACKGROUND

The present disclosure relates to a tracheal tube.

As a related art, a tracheostomy tube is known as a tracheal tube that directly connects the outside of a body and the inside of a trachea to facilitate breathing for a patient for which spontaneous breathing is difficult. To the tracheostomy tube, a cuff that can be deflated and inflated is attached on the outer circumferential surface of the tube main body. The tracheostomy tube is inserted into the trachea of the patient, with the cuff deflated, and then the cuff is inflated. This causes the cuff to get contact with the tracheal inner wall, so that the tracheostomy tube is indwelled in the trachea (for example, refer to Japanese Patent No. 5271898 and Japanese Patent Laid-open No. 2000-167060).

### SUMMARY

When the cuff is inflated in the trachea of a patient and the tracheal tube is used, foreign objects such as sputum, saliva, blood, and accidentally-swallowed object accumulate on the trachea upstream side (on the jaw side). The foreign objects that accumulate can be removed by being sucked from a lumen made in the tracheal tube for example. However, in some cases, wrinkles are formed in the outer surface of the cuff when the cuff is inflated in the trachea and a gap due to the wrinkle is generated between the outer surface of the cuff and the tracheal inner wall of the patient. The generation of the gap due to the wrinkle causes a possibility that a foreign object flows from this gap toward the trachea downstream side to flow into the lung. Due to the foreign object flowing into the lung in this manner, the risk that pneumonia develops in the patient increases.

Furthermore, in the state in which the cuff is inflated in the trachea, pressure from the cuff is applied to the place in contact with the cuff in the tracheal inner wall and there is a possibility that inflammation occurs at this place in contact with the cuff depending on the case.

An object of the present disclosure is to provide a tracheal tube that allows improvement in safety in view of the aforesaid problems.

A tracheal tube as a first mode of the present disclosure includes a tube main body and a cuff that is mounted to the tube main body and has a barrel portion capable of being deflated and inflated. A covering region that covers a tracheal inner wall of a trachea in a state in which the barrel portion is inflated in the trachea has a sealing part that gets contact with the tracheal inner wall seamlessly in the circumferential direction of the barrel portion. The width of at least part of the sealing part is smaller than 1 mm.

As one embodiment of the present disclosure, it is preferable that the membrane thickness of the barrel portion be smaller than 0.15 mm.

As one embodiment of the present disclosure, it is preferable that the barrel portion be composed of a material having a Young's modulus equal to or lower than 0.34 MPa.

As one embodiment of the present disclosure, it is preferable that the barrel portion be rotationally symmetric about a predetermined axis in the inflated state and have a taper part whose diameter around the predetermined axis increases from one end side of the predetermined axis toward the other end side.

According to the present disclosure, a tracheal tube that allows improvement in safety can be provided.

### BRIEF DESCRIPTION OF THE DRAWINGS

FIG. 1 is a diagram showing the state in which a tracheal tube according to one embodiment of the present disclosure is indwelled in a trachea;

FIG. 2 is a perspective view showing solely a tube main body in a tracheal tube shown in FIG. 1;

FIG. 3 is a lateral view showing the outline of a cuff included in the tracheal tube shown in FIG. 1;

FIG. 4 is a diagram showing one example of a covering region recorded on a laminate film, obtained by an experiment using the tracheal tube having the cuff shown in FIG. 3;

FIGS. 5A and 5B are diagrams schematically showing the relationship between a wrinkle part and a seamless sealing part in a covering region;

FIGS. 6A and 6B are diagrams showing two modification examples of the shape of the cuff included in the tracheal tube;

FIG. 7 is a diagram showing one example of a section of the state in which a distal connected part is fusion-bonded in such a manner as to extend to the distal side relative to a taper part and a proximal connected part is folded into the side of the taper part and is fusion-bonded; and

FIG. 8 is a flowchart showing one example of a method for mounding the cuff to the tracheal tube.

### DETAILED DESCRIPTION OF THE PREFERRED EMBODIMENT

A tracheal tube according to one embodiment of the present disclosure will be described below with reference to the drawings. A component common in the respective drawings is given the same symbol.

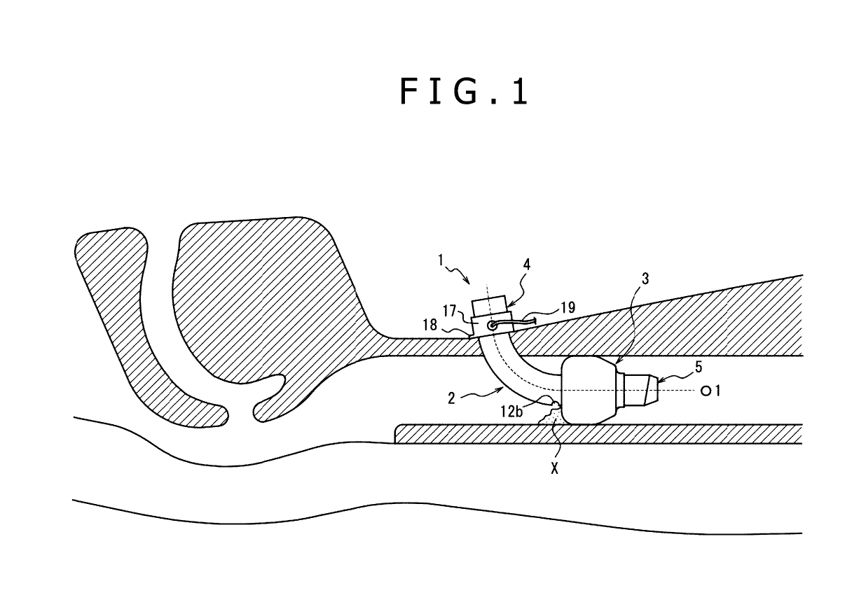

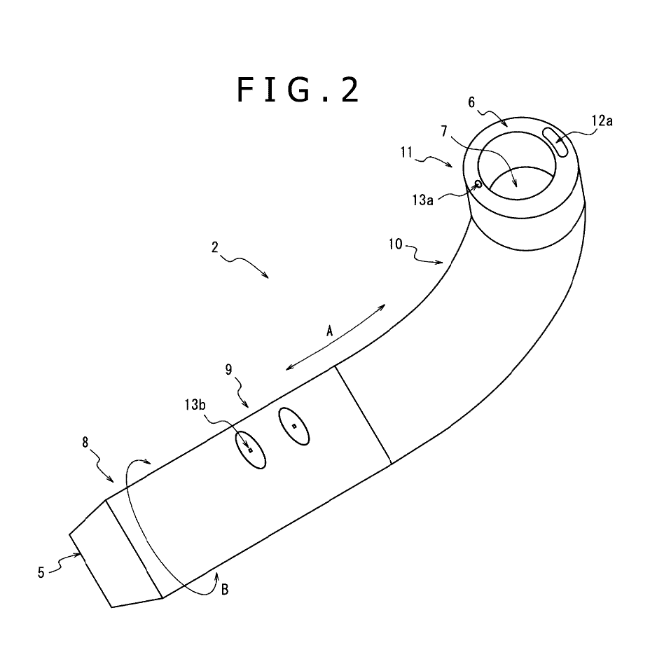

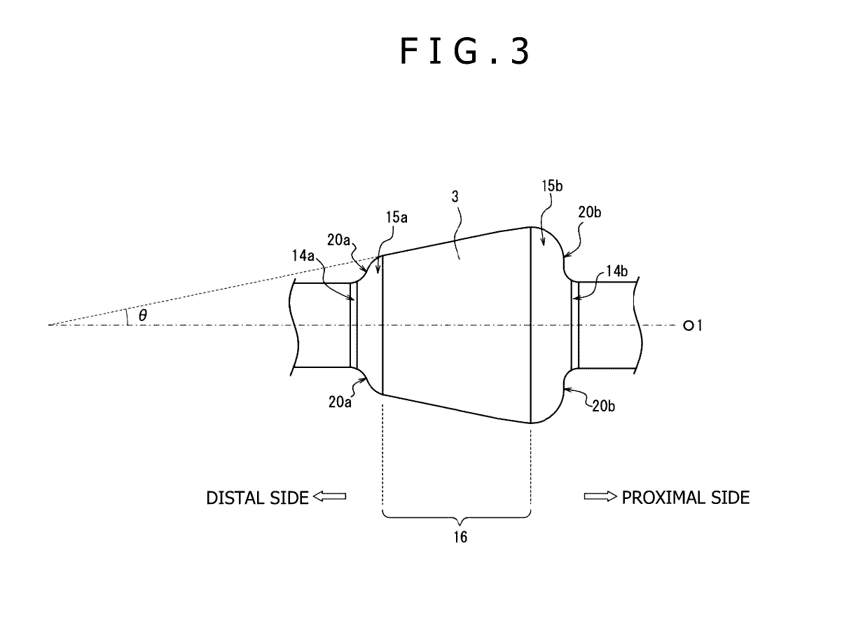

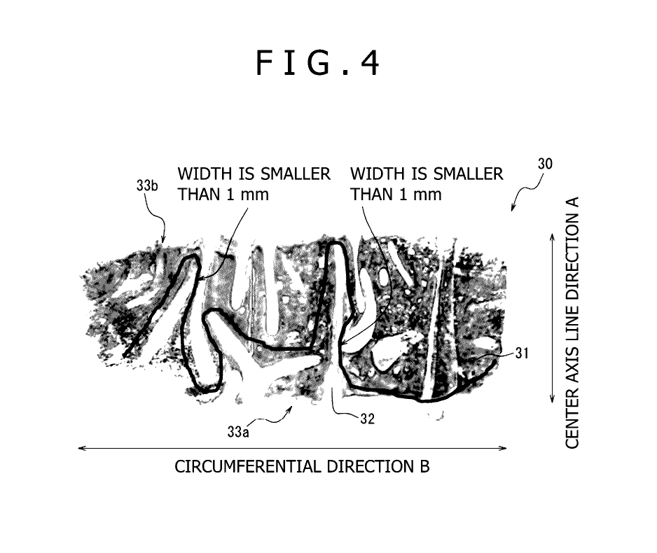

FIG. 1 is a diagram showing the state in which a tracheal tube 1 according to the one embodiment of the present disclosure is indwelled in a trachea. FIG. 2 is a perspective view showing solely a tube main body 2 in the tracheal tube 1 shown in FIG. 1. FIG. 3 is a lateral view showing the outline of a cuff 3 included in the tracheal tube 1 shown in FIG. 1. FIG. 4 is a diagram showing one example of a covering region recorded on a laminate film, obtained by an experiment using the tracheal tube 1 having the cuff 3 shown in FIG. 3.

As shown in FIG. 1, the tracheal tube 1 includes the tube main body 2, the cuff 3 that is attached onto the outer circumferential surface of this tube main body 2 and can be deflated and inflated, and a flange member 4 mounted to one end portion of the tube main body 2.

In the tube main body 2, a hollow part 7 that penetrates from a distal end 5 to a proximal end 6 in the extension direction of a center axis line O1 (hereinafter, referred to simply as “center axis line direction A”) of the outer circumferential surface of the tube main body 2 is marked out. In the state in which the tracheal tube 1 is inserted from the outside into the trachea and is indwelled, an airway is ensured by this hollow part 7. Note that the distal end 5 of the tube main body 2 is the distal end of the tube main body 2 and is one end located on the tracheal bifurcation side in the state in which the tracheal tube 1 is indwelled in the trachea. Furthermore, the proximal end 6 is the proximal end of the tube main body 2 and is the other end located on the jaw side in the state in which the tracheal tube 1 is indwelled in the trachea.

The tube main body 2 includes a distal portion 8 including the distal end 5, a cuff-mounted portion 9 that is continuous on the side of the proximal end 6 in the distal portion 8 in the center axis line direction A and has an outer circumferential surface onto which the cuff 3 is attached, a curving portion 10 that is continuous on the side of the proximal end 6 in this cuff-mounted portion 9, and a proximal portion 11 that is continuous on the side of the proximal end 6 in this curving portion 10 and includes the proximal end 6. In other words, the distal portion 8 of the tube main body 2 is linked to the proximal portion 11 with the intermediary of the cuff-mounted portion 9 and the curving portion 10. Note that the flange member 4 is mounted to the proximal portion 11.

In the wall of the tube main body 2 between the outer circumferential surface of the tube main body 2 and the inner circumferential surface that marks out the hollow part 7 of the tube main body 2, two hollow parts that extend along the center axis line O1 are marked out. Specifically, the tube main body 2 has a first lumen and a second lumen that are formed in the wall and extend along the center axis line O1 from a first proximal opening 12a and a second proximal opening 13a marked out in the proximal surface. Note that, although the first lumen and the second lumen that are marked out in the wall and have small diameters are also hollow parts, they are referred to as “lumen” here for differentiation from the large-diameter hollow part 7 for ensuring an airway for convenience of description.

The first lumen extends from the first proximal opening 12a in the proximal surface to a predetermined position on the side of the proximal portion 11 relative to the cuff 3 and the cuff-mounted portion 9, and communicates with the outside of the tube main body 2 through a suction port 12b that is formed at the predetermined position and penetrates to the outer circumferential surface of the tube main body 2. Note that the suction port 12b in the present embodiment is a suction port and is formed in the curving portion 10 as the position on the side of the proximal portion 11 relative to the cuff 3 and the cuff-mounted portion 9. The first lumen sucks and removes foreign objects X such as sputum, saliva, blood, and accidentally-swallowed object that accumulate on the trachea upstream side (on the jaw side) relative to the cuff 3 in the state of being indwelled in the trachea.

The second lumen extends from the second proximal opening 13a in the proximal surface to the position of the cuff 3 and the cuff-mounted portion 9, and communicates with the outside through a flow path 13b that is formed at the position and penetrates to the outer circumferential surface of the tube main body 2. Therefore, for example by using a syringe or the like, fluid such as air is supplied into the space (annular space) marked out by the outer circumferential surface of the cuff-mounted portion 9 and the inner surface of the cuff 3 from the second proximal opening 13a of the second lumen through the flow path 13b. Thereby, the cuff 3 can be inflated by this supplied fluid. Furthermore, for the cuff 3 in the inflated state, the fluid is sucked from the annular space through the flow path 13b and the second proximal opening 13a of the second lumen. This can deflate the cuff 3. As above, the second lumen is a lumen used for deflating and inflating the cuff 3 and will be referred to as the “cuff lumen” hereinafter.

As the constituent material of the tube main body 2, the following various kinds of resin can be used for example: silicone, polyvinyl chloride, polyethylene, polypropylene, cyclic polyolefin, polystyrene, poly-(4-methylpentene-1), polycarbonate, acrylic resin, acrylonitrile-butadiene-styrene copolymer, polyester such as polyethylene terephthalate, butadiene-styrene copolymer, and polyamide (for example, nylon 6, nylon 6/6, nylon 6/10, nylon 12). It is preferable to use, among them, a resin such as polyvinyl chloride, polypropylene, cyclic polyolefin, polyester, or poly-(4-methylpentene-1) in terms of easiness of shaping.

The flange member 4 is mounted to the proximal portion 11 (see FIG. 2 and so forth) of the tube main body 2 as shown in FIG. 1. When the tube main body 2 is inserted from the outside of the body into the trachea and the tracheal tube 1 is indwelled, the flange member 4 abuts against skin to fix the distal portion 8 at a proper position in the trachea. The proximal portion 11 of the tube main body 2 is internally inserted into the flange member 4. The flange member 4 includes a tubular part 17 that is mounted to the tube main body 2 by being fit to the tube main body 2 and has a circular cylindrical shape and a plate-shaped flange part 18 that protrudes from the outer wall of this tubular part 17 toward the outside in the radial direction and abuts against skin in the state in which the tracheal tube 1 is indwelled.

The first lumen communicates with the outside of the tracheal tube 1 on the proximal side of the tracheal tube 1 through a corresponding communication hole formed in the tubular part 17. Suction of the foreign object X such as an accidentally-swallowed object is carried out by connecting a syringe, a suction pump, or the like to one end of a suction tube 19 having the other end fit to the communication hole of the tubular part 17 exposed outside the body and performing suction.

Furthermore, the cuff lumen communicates with the outside of the tracheal tube 1 on the proximal side of the tracheal tube 1 through a corresponding communication hole formed in the tubular part 17. Therefore, if a syringe or the like is connected to one end of a tube for the cuff having the other end fit to the communication hole of the tubular part 17 exposed outside the body, supply and suction of fluid to and from the annular space of the cuff 3 can be carried out by operation of the syringe or the like existing outside the body and thereby inflation and deflation of the cuff 3 can be operated.

The flange member 4 can be formed by a material similar to that of the tube main body 2 for example.

The cuff 3 is attached onto the outer circumferential surface of the cuff-mounted portion 9 of the tube main body 2 and can be inflated through the above-described cuff lumen of the tube main body 2 by the pressure of the fluid supplied into the annular space marked out by the outer circumferential surface of the cuff-mounted portion 9 and the inner surface of the cuff 3. Therefore, in attempting to insert the tracheal tube 1 from the outside into the trachea and indwell the tracheal tube 1 at a predetermined position, the fluid is supplied into the annular space through the cuff lumen of the tube main body 2 and the cuff 3 is inflated. Thereby, the outer surface of the inflated cuff 3 is brought into tight contact with the tracheal inner wall and the cuff 3 is clamped by the inner circumferential wall surface of the trachea by the friction force between the outer surface of the cuff 3 and the tracheal inner wall, and so forth. In addition, the cuff 3 occludes the trachea around the tube main body 2. For this reason, the position of the cuff 3 in the trachea is fixed and the tracheal tube 1 can be indwelled at the above-described predetermined position.

Furthermore, in withdrawing the tracheal tube 1 from the inside of the trachea to the outside and in adjusting the position at which the tracheal tube 1 is indwelled, and so forth, the fluid in the annular space is sucked by using the cuff lumen and the cuff 3 is deflated. This allows the tube main body 2 of the tracheal tube 1 to move in the trachea along the trachea.

In the cuff 3, the end edge part on the side of the proximal end 6 in the center axis line direction A (corresponding to a proximal connected part 14b to be described later) and the end edge part on the side of the distal end 5 (corresponding to a distal connected part 14a to be described later) are each joined onto the outer circumferential surface of the cuff-mounted portion 9 by welding, high-frequency fusion bonding, or the like across the whole region of a circumferential direction B (see FIG. 2) of the tube main body 2. This forms the above-described annular space by the inner surface of the cuff 3 and the outer circumferential surface of the cuff-mounted portion 9.

A more detailed description will be made about the cuff 3 with reference to FIG. 3. In FIG. 3, only the cuff 3 and a part near the cuff 3 in the tube main body 2 to which the cuff 3 is attached are shown and the state in which the cuff 3 is inflated at a certain pressure (e.g. 16 to 25 mmHg) is shown. That is, the cuff 3 becomes the state shown in FIG. 3 when being inflated without being inserted into a trachea.

The cuff 3 has the distal connected part 14a and the proximal connected part 14b fusion-bonded to the tube main body 2 on the side of the distal end 5 and on the side of the proximal end 6. Furthermore, the cuff 3 has a barrel portion that can be inflated and deflated. In the present embodiment, the barrel portion has a taper part 16 and coupling parts. The coupling part couples the taper part 16 with the distal connected part 14a or the proximal connected part 14b. The coupling part that couples the taper part 16 and the distal connected part 14a will be referred to as a distal coupling part 15a particularly, and the coupling part that couples the taper part 16 and the proximal connected part 14b will be referred to as a proximal coupling part 15b particularly. The distal coupling part 15a has a gentle curved surface including an inflection point 20a in side view of the cuff 3. Similarly, the proximal coupling part 15b has a gentle curved surface including an inflection point 20b in side view of the cuff 3.

In the cuff 3, the outer shape of the taper part 16 formed between the distal connected part 14a and the proximal connected part 14b is a substantially conical trapezoid shape that has the distal connected part 14a and the proximal connected part 14b on the upper base side and the lower base side, respectively, and has the center axis line O1 as the center axis. Specifically, the taper part 16 is rotationally symmetric about the center axis line O1 and, in side view, has a taper shape in which the diameter of the outer surface increases from the side of the distal end 5 toward the side of the proximal end 6, with the center axis line O1 being the center of the diameter. In the example shown in FIG. 3, the distal connected part 14a and the proximal connected part 14b are fusion-bonded to the tube main body 2 in such a manner as to extend to the distal side and the proximal side, respectively, relative to the taper part 16. An angle (taper angle) θ formed by the taper part 16 in side view of the cuff 3 with the center axis line O1 is 12°.

In the taper part 16, the length along the center axis line O1 (equivalent to the height of the conical trapezoid), i.e. the interval between the coupling parts (cuff width), can be decided as appropriate and is 26 mm in the present embodiment. Furthermore, the maximum diameter (cuff diameter) of the circle formed by the outer surface of the taper part 16 in a surface orthogonal to the center axis line O1 can be decided as appropriate according to the size of the trachea of a patient for example and is e.g. 26 mm in the present embodiment. It is preferable that this maximum diameter be such a diameter that, when the cuff 3 is inflated, the tracheal tube 1 is indwelled in the trachea of a patient due to application of a predetermined proper pressure to the tracheal inner wall from the outer surface of the cuff 3.

The material of the membrane forming the outer surface of the cuff 3 in the present embodiment is soft polyvinyl chloride and has a Young's modulus of 0.34 MPa. Note that, as the material of the membrane forming the outer surface of the cuff 3, polyurethane, polyethylene, polypropylene, polyester, ethylene-vinyl acetate copolymer (EVA), silicone, or a material that is obtained by mixing any materials among them and has flexibility may be used for example. Furthermore, the membrane forming the outer surface of the cuff 3 in the present embodiment has a membrane thickness of 0.06 mm.

Next, an experiment made by the inventor with use of the tracheal tube 1 according to the present embodiment will be described. The inventor used the tracheal tube 1 according to the present embodiment for the trachea of a subject and made the experiment relating to the state of the contact between the cuff 3 and the trachea of the subject in the use state.

Specifically, first the inventor stuck a laminate film to the inner surface of the trachea by inserting the laminate film turned to a tubular shape into the trachea of the subject and to the position with which the inflated cuff 3 got contact when the tracheal tube 1 was used. As the laminate film, a film (thickness 0.1 mm) exclusively for the laminator, made by ASKA CORPORATION, was used. Next, the tracheal tube 1 was inserted into the trachea of the subject in the state in which the cuff 3 on which paint was applied on the outer surface was deflated, and the cuff 3 was inflated at a certain pressure (e.g. 16 to 25 mmHg). At this time, the paint applied on the outer surface of the cuff 3 adhered to the part in contact with the outer surface of the barrel portion of the cuff 3 in the laminate film stuck to the inner surface of the trachea. On the other hand, the part at which a wrinkle was generated in the outer surface of the barrel portion of the cuff 3 did not get contact with the laminate film and therefore the paint on this part did not adhere to the laminate film. Then, the cuff 3 was deflated and the tracheal tube 1 was withdrawn from the trachea. Thereafter, the laminate film was peeled off from the trachea of the subject.

FIG. 4 is a diagram showing one example of the covering region recorded on the laminate film, obtained by the above-described experiment. FIG. 4 shows the laminate film that was withdrawn from the trachea of the subject and then was cut along the center axis line direction A to be extended. Here, a covering region 30 is a region that covers the tracheal inner wall in the state in which the barrel portion of the cuff 3 is inflated in the trachea of the subject. The covering region 30 has a sealing part 31 as the part that gets contact with the tracheal inner wall in the barrel portion and a wrinkle part 32 that covers the tracheal inner wall but does not get contact with the tracheal inner wall.

The sealing part 31 is the part that gets contact with the tracheal inner wall in the barrel portion and therefore appears as a region to which the paint adheres in the laminate film shown in FIG. 4. The sealing part 31 is a region where the barrel portion is brought into tight contact with the tracheal inner wall and thus a foreign object such as an accidentally-swallowed object does not flow into the sealing part 31.

The wrinkle part 32 is a region formed of wrinkles that do not get contact with the tracheal inner wall and therefore appears as a region to which the paint does not adhere in the laminate film shown in FIG. 4. The wrinkle part 32 is formed due to that the outer surface of the barrel portion is not fully inflated and wrinkles are formed in the outer surface of the barrel portion. Therefore, the wrinkle part 32 has a space between the barrel portion and the tracheal inner wall. For this reason, there is a possibility that a foreign object such as an accidentally-swallowed object flows into the wrinkle part 32.

As shown in FIG. 4, the sealing part 31 extends across the whole of the laminate film in the circumferential direction (same direction as the circumferential direction B of the tube main body 2 in the tracheal tube 1). In the present embodiment, the covering region 30 has the sealing part 31 that is seamless in the circumferential direction of the barrel portion (same direction as the circumferential direction B of the tube main body 2 in the tracheal tube 1) in particular. In FIG. 4, one example of the seamless sealing part 31 is shown by a heavy line. The seamless sealing part 31 formed in the covering region 30 is not limited to the sealing part 31 indicated by the heavy line in FIG. 4 and includes the sealing part 31 continuously formed seamlessly in the circumferential direction B in the sealing part 31 formed in the covering region 30. That is, the seamless sealing part 31 refers to a place where unicursal drawing is possible in the circumferential direction B in the sealing part 31.

Due to that the covering region 30 has the seamless sealing part 31 as above, the sealing part 31, into which a foreign object does not flow, is continuously formed seamlessly in the circumferential direction B. This eliminates the flowing of a foreign object from the proximal side of the cuff 3 into the distal side. For this reason, the risk of the flowing of the foreign object into the lung is reduced and the risk that pneumonia develops in the patient is reduced. In this manner, the safety of the tracheal tube 1 is enhanced.

In the present embodiment, in the seamless sealing part 31, the width is smaller than 1 mm at least partly. Here, that the width is smaller than 1 mm at least partly means that the smallest width of the sealing part 31 formed between wrinkles or between a wrinkle and a proximal rim part 33b or a distal rim part 33a of the covering region 30 is smaller than 1 mm. In the example shown in FIG. 4, in the direction perpendicular to (normal direction of) the tangential direction of a unicursal line, the width of at least part of the sealing part 31 formed between wrinkles or between a wrinkle and the proximal rim part 33b or the distal rim part 33a of the covering region 30 is smaller than 1 mm. In FIG. 4, a place where the width is smaller than 1 mm in the seamless sealing part 31 is indicated by an arrow.

In the sealing part 31 whose width is smaller than 1 mm as above, the area of contact with the tracheal inner wall is also small. For this reason, the region to which pressure is applied from the cuff is also small and the possibility that inflammation occurs in the tracheal inner wall is reduced. In this manner, the safety of the tracheal tube 1 is enhanced. Note that it is preferable that the width of the whole of the seamless sealing part 31 be smaller than 1 mm, i.e. the maximum width be smaller than 1 mm, to reduce the possibility that inflammation occurs in the tracheal inner wall.

To facilitate formation of the place where the width is smaller than 1 mm in the seamless sealing part 31, it is preferable that the barrel portion be composed of a soft material. This is because, when the material of the barrel portion is softer, the barrel portion is deformed more readily and a region with a smaller width is readily formed in the sealing part 31. Therefore, although the case in which the membrane of the barrel portion is composed of a material having a Young's modulus of 0.34 MPa is described in the aforesaid embodiment, it is preferable that the membrane of the barrel portion be composed of a material having a Young's modulus equal to or lower than 0.34 MPa.

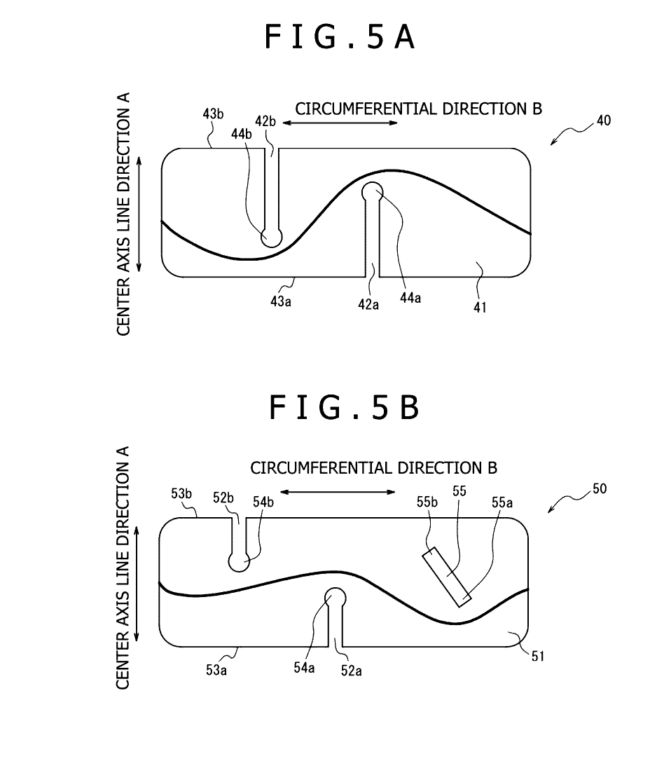

In the present embodiment, the wrinkle part 32 appears in a section obtained by cutting the laminate film (covering region 30) by an arbitrary surface orthogonal to the center axis line O1 of the tube main body 2. The cutting by an arbitrary surface orthogonal to the center axis line O1 of the tube main body 2 means cutting in the direction parallel to the circumferential direction B in FIG. 4. That the wrinkle part 32 appears in a section obtained by cutting the covering region 30 by an arbitrary surface orthogonal to the center axis line O1 of the tube main body 2 means that the seamless sealing part 31 does not extend continuously in the direction orthogonal to the center axis line O1 of the tube main body 2 across the whole region of the circumferential direction B. A further description will be made about the relationship between the wrinkle part 32 and the seamless sealing part 31 in the present embodiment with reference to FIGS. 5A and 5B.

FIGS. 5A and 5B are diagrams schematically showing the relationship between the wrinkle part and the seamless sealing part in a covering region. A covering region 40 shown in FIG. 5A has a first wrinkle 42a extending in the direction from a distal rim part 43a to a proximal rim part 43b of the covering region 40 and a second wrinkle 42b extending in the direction from the proximal rim part 43b to the distal rim part 43a. The first wrinkle 42a does not reach the proximal rim part 43b and the second wrinkle 42b does not reach the distal rim part 43a. That is, the first wrinkle 42a and the second wrinkle 42b do not communicate between the distal rim part 43a and the proximal rim part 43b. Furthermore, an end part 44a of the first wrinkle 42a on the side of the proximal end 6 is located on the side of the proximal end 6 relative to an end part 44b of the second wrinkle 42b on the side of the distal end 5. Due to that the first wrinkle 42a and the second wrinkle 42b are disposed as shown in FIG. 5A, at least one of the first wrinkle 42a and the second wrinkle 42b appears in a section when the covering region 40 is cut by an arbitrary surface orthogonal to the center axis line O1.

A heavy line shown in the covering region 40 of FIG. 5A is one example of a seamless sealing part 41. When the first wrinkle 42a and the second wrinkle 42b are disposed as shown in FIG. 5A, the seamless sealing part 41 does not form a line parallel to the circumferential direction B but forms a line waving in the center axis line direction A, i.e. a serpentine line.

A covering region 50 shown in FIG. 5B has a first wrinkle 52a extending in the direction from a distal rim part 53a to a proximal rim part 53b of the covering region 50, a second wrinkle 52b extending in the direction from the proximal rim part 53b to the distal rim part 53a, and a third wrinkle 55 that includes neither the distal rim part 53a nor the proximal rim part 53b. The first wrinkle 52a does not reach the proximal rim part 53b and the second wrinkle 52b does not reach the distal rim part 53a. That is, none of the first wrinkle 52a, the second wrinkle 52b, and the third wrinkle 55 communicate between the distal rim part 53a and the proximal rim part 53b. In the example shown in FIG. 5B, differently from the example shown in FIG. 5A, an end part 54a of the first wrinkle 52a on the side of the proximal end 6 is located on the side of the distal end 5 relative to an end part 54b of the second wrinkle 52b on the side of the distal end 5. Furthermore, an end part 55a of the third wrinkle 55 on the side of the distal end 5 is located on the side of the distal end 5 relative to the end part 54a of the first wrinkle 52a on the side of the proximal end 6. Moreover, an end part 55b of the third wrinkle 55 on the side of the proximal end 6 is located on the side of the proximal end 6 relative to the end part 54b of the second wrinkle 52b on the side of the distal end 5. Due to that the first wrinkle 52a, the second wrinkle 52b, and the third wrinkle 55 are disposed as shown in FIG. 5B, at least one of the first wrinkle 52a, the second wrinkle 52b, and the third wrinkle 55 appears in a section when the covering region 50 is cut by an arbitrary surface orthogonal to the center axis line O1.

A heavy line shown in the covering region 50 of FIG. 5B is one example of a seamless sealing part 51. Also when the first wrinkle 52a, the second wrinkle 52b, and the third wrinkle 55 are disposed as shown in FIG. 5B, the seamless sealing part 51 does not form a line parallel to the circumferential direction B but forms a serpentine line similarly to the case of the example shown in FIG. 5A.

As above, the seamless sealing part forms the serpentine line due to that the covering region has the wrinkle part that appears in a section obtained by cutting by an arbitrary surface orthogonal to the center axis line O1 of the tube main body 2. For this reason, the pressure applied from the barrel portion to the tracheal inner wall is readily dispersed in the center axis line direction A of the covering region and the burden on the tracheal inner wall is readily alleviated. Therefore, reduction in the burden on the patient is facilitated.

Note that, in the example described in FIG. 5A, the seamless sealing part 41 forms a line that meanders more greatly when the end part 44a of the first wrinkle 42a on the side of the proximal end 6 is closer to the proximal rim part 43b and the end part 44b of the second wrinkle 42b on the side of the distal end 5 is closer to the distal rim part 43a. For this reason, the pressure applied from the barrel portion to the tracheal inner wall is readily dispersed in the center axis line direction A more widely in the covering region and the burden on the tracheal inner wall is alleviated more readily.

Although the present disclosure is described based on the drawings and embodiment examples, it should be noted that it is easy for those skilled in the art to make various modifications and alterations based on the present disclosure. Therefore, it should be noted that these modifications and alterations are included in the scope of the present disclosure.

For example, in the aforesaid embodiment, the case in which the cuff 3 has the barrel portion with a taper shape is described. However, the shape of the barrel portion is not limited to the taper shape. The cuff 3 can have the barrel portion with an arbitrary shape that can produce effects of the disclosure of the present application.

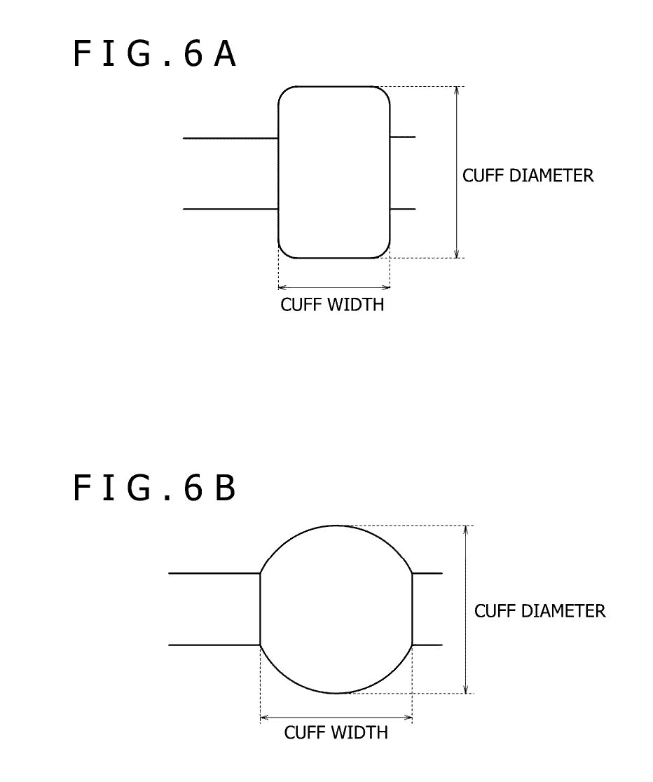

FIGS. 6A and 6B are diagrams showing two modification examples of the shape of the cuff 3 included in the tracheal tube 1. A first modification example has a substantially circular column shape whose outer diameter (cuff diameter) is larger than the diameter of the tube main body as shown in FIG. 6A, and this type will be referred to as the tire type hereinafter. The cuff of the tire type has a straight line part parallel to the tube main body in side view in the barrel portion of the cuff. A second modification example has a substantially spherical shape as the outer shape as shown in FIG. 6B, and this type will be referred to as the spherical type hereinafter. In the cuff of the spherical type, the barrel portion of the cuff has a curved surface in side view.

Also in the cases in which the shape of the barrel portion is the tire type and the spherical type, the same effects as the tracheal tube 1 of the aforesaid embodiment described by taking the barrel portion having the taper shape as an example are achieved if the covering region 30 has the seamless sealing part 31 and the width of at least part of the sealing part 31 is smaller than 1 mm.

Furthermore, in the aforesaid embodiment, it is explained that the distal connected part 14a and the proximal connected part 14b are fusion-bonded to the tube main body 2 in such a manner as to extend to the distal side and the proximal side, respectively, relative to the taper part 16. However, the form of the fusion bonding of the distal connected part 14a and the proximal connected part 14b is not limited thereto. For example, the cuff 3 and the tube main body 2 may be fusion-bonded by folding the distal connected part 14a and the proximal connected part 14b into the side of the taper part 16 and fusion-bonding the distal connected part 14a and the proximal connected part 14b to the tube main body 2 in the barrel portion. Furthermore, only one of the distal connected part 14a and the proximal connected part 14b may be folded into the side of the taper part 16.

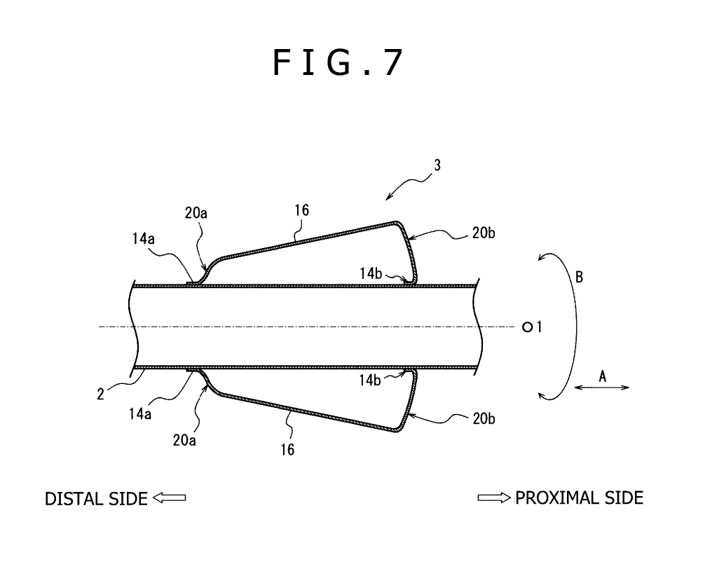

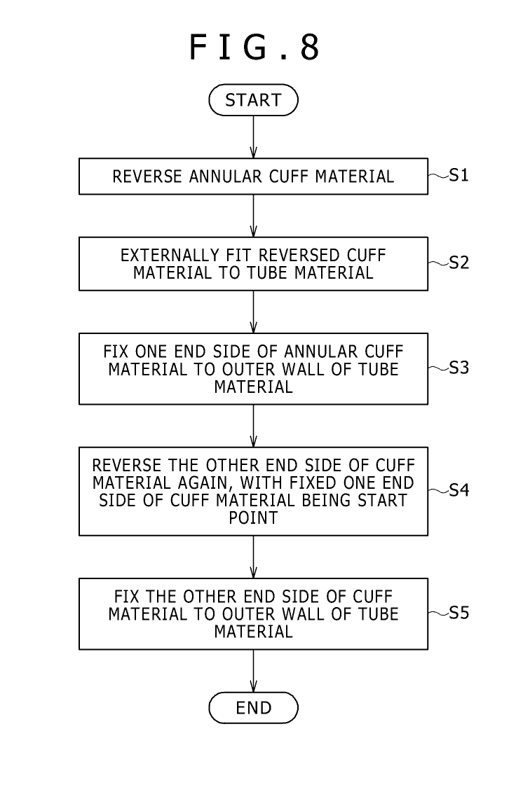

FIG. 7 is a diagram showing one example of a section of the state in which the distal connected part 14a is fusion-bonded in such a manner as to extend to the distal side relative to the taper part 16 and the proximal connected part 14b is folded into the side of the taper part 16 and is fusion-bonded. The mounting method of the cuff 3 shown in FIG. 7 will be described with reference to FIG. 8.

As shown in FIG. 7, the mounting method of the cuff 3 includes a step S1 of reversing an annular cuff material, a step S2 of inserting a tube material into the reversed cuff material to externally fit the reversed cuff material to the tube material, and a step S3 of fixing one end side of the annular cuff material to the outer wall of the tube material. The mounting method further includes a step S4 of reversing the other end side of the cuff material again, with the fixed one end side of the cuff material being the start point of the reversing, and a step S5 of fixing the other end side of the cuff material to the outer wall of the tube material. Note that the tube material means the completed tube main body 2 or what serves as the base of the tube main body 2. Furthermore, the annular cuff material means a component that forms the cuff 3 of the tracheal tube 1 by being mounted to the tube material and serves as the base of the cuff 3. Moreover, the annular cuff material includes a large-diameter part to become the barrel portion and end edge parts that are continuous with both sides of this large-diameter part and have a diameter smaller than that of the large-diameter part. The respective steps will be described in detail below.

In the step S1, the annular cuff material is reversed so that the surface to become the inner wall of the barrel portion in the large-diameter part may be exposed to the outside. However, the step S1 can be omitted if such a distinction between the front and back sides does not exist in the annular cuff material or if the cuff material has been already reversed.

In the step S2, the reversed annular cuff material is externally fitted from one end side of the tube material (for example, one end side to become the proximal end 6 of the tube main body 2). Note that the reversed annular cuff material may be externally fitted from the other end side of the tube material (for example, one end side to become the distal end 5 of the tube main body 2).

In the step S3, the end edge part to become the proximal connected part 14b as the end edge part on one end side of the annular cuff material externally fitted to the tube material is fixed to the outer wall of the tube material by fusion bonding or the like. Note that, when being fixed to the outer wall of the tube material, the end edge part on the one end side of the cuff material is in the state of extending to the outside of the large-diameter part in the center axis line direction of the tube material (same direction as the center axis line direction A of the tube main body 2). Furthermore, although being the end edge part to become the proximal connected part 14b, the end edge part on the one end side fixed to the tube material is located on the distal side of the tube material relative to the large-diameter part, i.e. on the side to become the side of the distal end 5 of the tube main body 2, because the cuff material is in the reversed state when this end edge part on the one end side is fixed to the outer wall of the tube material.

In the step S4, the other end side of the cuff material is reversed again, with the fixed end edge part on the one end side of the cuff material being the start point of the reversing. That is, the other end side is reversed so that the surface to become the outer wall of the barrel portion in the large-diameter part may be exposed to the outside. More specifically, with the proximal connected part 14b being the start point of the reversing, the end edge part to become the distal connected part 14a as the end edge part on the other end side of the annular cuff material and the large-diameter part to become the barrel portion are reversed. This causes the proximal connected part 14b to become the state of being folded into the side of the large-diameter part to become the barrel portion in the center axis line direction of the tube material. Meanwhile, the end edge part to become the distal connected part 14a becomes the state of extending to the outside of the large-diameter part to become the barrel portion, specifically to the distal side of the tube material relative to the large-diameter part, in the center axis line direction of the tube material.

In the step S5, the end edge part to become the distal connected part 14a as the end edge part on the other end side of the cuff material is fixed to the outer wall of the tube material by fusion bonding or the like. This completes the fixing of the cuff material to the tube material.

In the above-described manner, the cuff 3 can be mounted to the tube material. Note that, although the mounting method of the cuff 3 is described with reference to FIG. 8 here, in the case of the cuff 3 shown in FIG. 3, it suffices to carry out a step of externally fitting an annular cuff material to become the cuff 3 to a tube material without reversing the cuff material and fixing one end side of the annular cuff material to the outer wall of the tube material and a step of fixing the other end side of the cuff material to the outer wall of the tube material. More specifically, both end edge parts that extend to the outside of the large-diameter part to become the barrel portion in the direction of the center axis line O1 of the tube material in the annular cuff material externally fitted to the tube material are fixed to the outer wall of the tube material by fusion bonding or the like, to form the distal connected part 14a and the proximal connected part 14b.

Furthermore, although it is explained that the membrane thickness of the barrel portion is 0.06 mm in the aforesaid embodiment, the membrane thickness is not limited thereto. It suffices for the membrane thickness of the barrel portion to be larger than 0 mm and be smaller than 0.15 mm for example. Note that it is preferable that the membrane thickness of the cuff 3 be larger than 0 mm and be equal to or smaller than 0.10 mm and it is more preferable that the membrane thickness be larger than 0 mm and be equal to or smaller than 0.08 mm.

Note that it is preferable that the membrane thickness of the barrel portion be small to facilitate formation of the place where the width is smaller than 1 mm in the seamless sealing part 31. This is because the barrel portion is deformed more readily and a region with a smaller width is readily formed in the sealing part 31 when the membrane thickness of the barrel portion is smaller.

Furthermore, although it is explained that the taper angle θ of the cuff 3 of the taper type is 12° in the aforesaid embodiment, the taper angle θ of the cuff of the taper type is not limited to 12°. The taper angle θ of the cuff of the taper type can be set to an arbitrary angle that allows formation of the seamless sealing part 31 in the circumferential direction of the barrel portion in the covering region 30, and can be set equal to or smaller than 40° for example. Note that, to form the seamless sealing part 31 in the circumferential direction of the barrel portion in the covering region 30, it is preferable to set the taper angle θ equal to or smaller than 25° and it is more preferable to set the taper angle θ equal to or larger than 10° and equal to or smaller than 12°.

As described above, the seamless sealing part 31 having a place where the width is smaller than 1 mm can be realized by setting the membrane thickness of the barrel portion in a range that is larger than 0 mm and is smaller than 0.15 mm, setting the material of the membrane in a range in which the Young's modulus is equal to or lower than 0.34 MPa, and setting the taper angle θ in a range equal to or smaller than 40° as appropriate for example.

The present disclosure relates to a tracheal tube.

Having described the preferred embodiment of the present disclosure with reference to the accompanying drawings, it is to be understood that the disclosure is not limited to the precise embodiment and that various changes and modifications could be effected therein by one skilled in the art without departing from the spirit or scope of the disclosure as defined by the appended claims.

What is claimed is:

1. A tracheal tube comprising:
a tube main body; and
a cuff that is mounted to the tube main body and has a barrel portion capable of being deflated and inflated, wherein a covering region that covers a tracheal inner wall of a trachea in a state in which the barrel portion is inflated in the trachea has a sealing part that gets contact with the tracheal inner wall seamlessly in a circumferential direction of the barrel portion, and width of at least part of the sealing part is smaller than 1 mm.

2. The tracheal tube according to claim 1, wherein membrane thickness of the barrel portion is smaller than 0.15 mm.

3. The tracheal tube according to claim 1, wherein the barrel portion is composed of a material having a Young's modulus equal to or lower than 0.34 MPa.

4. The tracheal tube according to claim 1, wherein the barrel portion is rotationally symmetric about a predetermined axis in the inflated state and has a taper part whose diameter around the predetermined axis increases from one end side of the predetermined axis toward the other end side.
